(19) United States
(12) Patent Application Publication
(10) Pub. No.: US 2023/0123388 A1
(43) Pub. Date: Apr. 20, 2023

(54) TRANSDERMAL AND/OR TOPICAL DELIVERY SYSTEM COMPRISING HYDROXYCHLOROQUINE AND/OR CHLOROQUINE

(71) Applicant: GLANIS PHARMACEUTICALS, INC., Vancouver (CA)

(72) Inventors: Fotios M. PLAKOGIANNIS, Whitestone, NY (US); Nisarg MODI, Jersey City, NJ (US)

(21) Appl. No.: 17/915,156

(22) PCT Filed: Apr. 13, 2021
(86) PCT No.: PCT/US2021/027051

§ 371 Date: Sep. 28, 2022

Related U.S. Application Data
(60) Provisional application No. 63/009,718, filed on Apr. 14, 2020.

(51) Int. Cl. A61K 9/70; A61K 31/4706

(52) U.S. Cl. CPC A61K 9/7084; A61K 31/4706

(57) ABSTRACT

A Transdermal Drug Delivery System (TDDS) of the reservoir or plaster or adhesive type for administrating Hydroxychloroquine and/or Chloroquine for the treatment of rheumatoid arthritis, lupus erythematosus, SARS CoV-2, porphyria cutanea tarda a for 1 day, 2 day, 3 day, 4 day, 5 day, 6 day and/or 7—day continuous application.

This International application claims benefit of U.S. Ser. No. 63/009,718 filed Apr. 14, 2020, the entirety of which is incorporated herein by reference.

### SPECIFICATION

### Background of the Invention

The present invention relates to transdermal drug delivery system (TDDS) of pharmaceutical compositions, which have a satisfactory in-vitro performance and good bioavailability. In particular, the transdermal pharmaceutical composition of Hydroxychloroquine/Chloroquine in the present invention includes either liquid or semi solid in a reservoir patch dosage form or matrix or adhesive in a plaster dosage form for treatment of rheumatoid arthritis, malaria, lupus erythematosus. SARS CoV-2 Infection, and porphyria Cutanea tarda for 1 Day, 2 Day, 3 Day, 4 Day, 5 Day, 6 Day and/or 7-day continuous application.

Rheumatoid arthritis (RA) is a chronic, inflammatory, autoimmune disorder. It is characterized by symmetric inflammation of synovial joints leading to progressive erosion of cartilage and bone. Upon untreated, the irreversible joint damage occurs within 2 years. During this disease, the body's own immune system attacks the lining of the membrane that surrounds the joints. 1-2% of world population is suffering from this disease. The occurrence of this disease is 3 times more in women than the men. This disease can occur at any age but the most patients are between 30 to 50 years of age. It can reduce the total life span of patient by 3 to 18 years. The average treatment cost in US is $6000/case/year¹.

Lupus erythematosus (LE) is another autoimmune disease which mainly targets women of childbearing age although it occurs at both extremes of age and in either sex. There are variable clinical presentation ranging from a skin rash uncomplaining by extra cutaneous stigmata to one comprising progressive multisystem disease².

Porphyria Cutanea tarda (PCT) is a type of porphyria or blood disorder that affects the skin. PCT is one of the most common type of porphyria. It is sometimes referred as a Vampire Disease because people with this condition often experience symptoms following exposure to sunlight. It affects female more than males. The disorder usually develops after the age of 30. PCT is a rare disorder; the occurrence is estimated to be approximately 1 case in every 10000 to 25,000 individuals in the general population³ Currently, American porphyria foundation (APF) recommends using 100 mg twice a week hydroxychloroquine. Which represents 28 mg/day. The APF recommendation dose can be developed transdermal. HCQ and/or CQ has been used off label due to unknown mechanism of action of this drug. The other reasons are no availability of suitable dosage form, tablets are not scored for division and the 100 mg tablet is not available in the market⁴.
Hydroxychloroquine (HCQ) is an immunomodulatory drug. Its sulfate salt, such as Plaquenil, is approved for the treatment of lupus erythematosus, rheumatoid arthritis, and malaria. Furthermore, HCQ and/or CQ gained interest as a potential therapeutic option for COVID-19 based on in vitro studies suggesting efficacy of HCQ and/or CQ against SARS-COv and SARS-COv-2⁵. Oral doses of the tablet range from 200 to 600 mg/day⁶. Pharmacokinetic studies reveal that the oral bioavailability is about 75% with rapid absorption kinetics. The drug is highly lipophilic and has a very large volume of distribution which results in a very long half-life (˜50 Hrs). Plasma levels of the drug can be variable with variable absorption kinetics⁷, but subsequent studies established that measurement of whole blood levels rather than plasma levels, and dosing based upon body weight considerably reduces this variability.
Hydroxychloroquine sulfate is a white, crystalline powder which is freely soluble in water and practically insoluble in alcohol, chloroform, and in ether. The molecular weight of hydroxychloroquine sulfate is 433.95⁶.

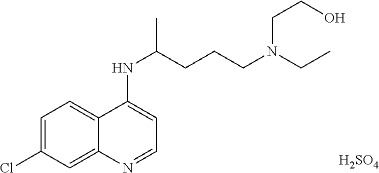 [SMILES: CCN(CCO)CCCC(C)Nc1ccnc2cc(Cl)ccc12 ; "H2SO4"]

The hydroxychloroquine base has a log P value of 3.6 with melting point of 90° C. The water solubility of base is 0.026 mg/ml⁹. The base may have very good solubility in organic solvents as compare to its salt due to higher log P value.
HCQ is currently available as a 200 mg oral tablet of sulfate salt of API, which is equivalent to 155 mg of the base form of API. The numerous studies concluded that IICQ peak concentration in blood was around 129.6 ng/ml while the plasma concentration was around 50.3 ng/ml in around 3 hours following 200 mg oral dose administration. Furthermore, in randomized cross over study, the bioavailability of HCQ was found to be 0.74 based on the 155 mg dose administration through oral and IV infusion^{10,11}.
HCQ showed moderate protein binding (˜40%) with albumin and apha1-acid glycoprotein. HCQ showed high volume of distribution because of deep tissue distribution^{12,13}, Animal study showed the high concentration of HCQ and/or CQ in the lungs, kidney, liver and spleen. In one of the animal studies, HCQ and/or CQ concentration was found to be 6-7 times higher in lung than the plasma¹⁴.
Previous research reported HCQ clearance to be 15.5 L/Hr. Most research also have reported the terminal half-life of 30-60 days based on blood concentration and ˜32 days based on the plasma concentration profile.¹⁰
Due to positive in-vitro studies on antiviral effect of HCQ and/or CQ, it gained interest as potential therapy against SARS-CoV-2. The anti-inflammatory action of HCQ and/or CQ is dependent on immunomodulation and the downstream production of cytokines. Furthermore, successful entry of SARS-CoV-2 into host cell is strongly dependent on angiotensin-converting enzymes-² (ACE-2) interaction with the viral spike protein. HCQ and/or CQ reduces the glycosylation of ACE-2, which inhibits the binding of SARS-CoV-2 spike protein to the cell surface and cell integration. Recent study showed that HCQ and/or CQ binds with the gangliosides, which inhibits communication between spike protein and the cell membrane and thus inhibiting viral entry to the cell¹⁵⁻¹⁸.
Rare but serious side effects have been reported, mostly with long term use. HCQ and/or CQ-induced acquired lysosomal storage disease causes some serious adverse effects, including myopathy and cardiomyopathy¹⁹. Most cases are caused by accumulation which can be augmented by CYP450 2C8 mutation²⁰. Due to its higher log Ko/w value HCQ and/or CQ easily permeates myocytes, in which it binds lysosomal phospholipids, leading to lysosomal accumulation of phospholipids. Furthermore, HCQ and/or CQ inhibits lysosomal enzymes by increasing the pH, which leads to accumulation of glycogen and phospholipids. The abnormal accumulation of metabolic products in lysosomal results into lysosomal storage disease, leading to myofibrillar disorganization, atrophy, and fibrosis, which may lead to cardiomyopathy^{21,22}.
HCQ and/or CQ affect myocardial depolarization and repolarization through cardiac K+ channel blockage causing QT/QTc prolongation, which is an indicator of an increase risk of drug-induced torsade de pointes (TdP). TdP is usually self-limiting but can degenerate into lethal ventricular fibrillation and cause sudden cardiac death²⁰.
Currently, the recommended dose of less than 5 mg/kg/day, HCQ and/or CQ is usually safe, although prolongation of the QT/QRS is rarely observed on a surface electrocardiogram⁶.
The oral administration of HCQ and/or CQ has been associated with toxicity and reports of various adverse events as described above which may lead to reduced dose or discontinuation of treatment.
Most drugs that exhibit adverse events are primarily due to the following reasons:
High peak plasma concentration after oral administration
Multiple oral dosing, which may result in higher plasma levels as a result of drug clearance.
Drugs with lower oral bioavailability require higher dose levels to achieve therapeutic blood levels which may result in toxicity.
Drugs with a small therapeutic window may have adverse events due to peak and valley profile.
   The therapeutic effect of HCQ and/or CQ with fewer side effects may be achieved by targeting a low continuous dose to achieve steady state plasma concentrations within the therapeutic window using transdermal delivery.

It is typical for transdermal drug delivery to achieve similar pharmacokinetic profiles as compared to an oral dose based on oral bioavailability and potentially reduce the overall exposure to the drug for very low oral bioavailable drugs. Currently, 15 drugs are commercially available as a transdermal delivery system and each of these has lower transdermal doses compared to their reference listed drugs through the oral dosage form. These products have shown bioequivalence to the innovator products. Furthermore, the drug applied transdermally bypasses the first pass metabolism which may result in a reduction in adverse events. Transdermal drug products continuously deliver the active molecule in plasma at the steady state plasma concentration throughout the application period, which prevents peaks and valleys associated with typical oral drug delivery.

Transdermal drug delivery can be advantageous over oral because it avoids first-past effects, and variations in absorption rates due intestinal activity and content. However, unlike the intestinal tract, transdermal delivery is limited by the high hydrophobic barrier function of the skin and a limited surface area for absorption. The best candidates for transdermal delivery are small molecular weight, lipophilic molecules that are extremely potent, requiring doses less than 25 mg/day. The half-life of the drug played a vital role during multiple dosing of transdermal system.

The longer half-life eliminates the lag time during the consequence dosing due to availability of API from the previous dosing.

### BRIEF SUMMARY OF THE INVENTION

The structure of reservoir TDDS as disclosed herein comprises an active substance, Hydroxychloroquine and/or chloroquine in the form of liquid, or semisolid or suspension in the pouch system. The pouch system contains impermeable backing layer, which covers the TDDS on the side averted from the skin and detachable protective layer containing release liner in contact with skin for controlled delivery of Hydroxychloroquine and/or chloroquine through the transdermal route.

The structure of matrix or plaster TDDS as disclosed herein comprises an active substance, Hydroxychloroquine and/or chloroquine, suspended or solubilize in the polymer or adhesive matrix, cover between impermeable backing layer and release liner and/or detachable protective layer. According to current invention, the active substance, Hydroxychloroquine and/or chloroquine itself is solubilized or suspended in the pressure-sensitive adhesive or polymer matrix, or an extra placebo pressure sensitive adhesive layer may be provided which enables fixation of the TDDS on the skin.

The detachable and protective layer during storage covers the release liner in reservoir TDDS and the pressure sensitive adhesive TDDS surface facing the skin and is detached before application.

The disclosure provides both TDDS designed as matrix system and or TDDS designed as reservoir membrane system.

The TDDS as disclosed herein can be used both in the form of reservoir system as well as in the form of matrix system. According to this invention reservoir system comprises a pouch formed from an impermeable backing, a rate controlling membrane, an adhesive peripheral ring, covered by a strippable protective backing. The impermeable backing is configured to provide a central volume, which contains a drug reservoir in the form of a semisolid or liquid having dissolved and suspended drug, therein. Although preferred embodiments utilize an adhesive peripheral ring outside the path of drug from the system to the skin but other means for maintaining the system on the skin can be employed. Such means include an in-line adhesive layer; adhesive overlays or other fastening means such as buckles, belts and elastic armbands is also contemplated.

The TDDS as disclosed herein can be manufactured in such a manner that this active substance, Hydroxychloroquine and/or chloroquine containing mixture is coated onto a suitable support, for example to a thermoplastic film provided with a silicone layer, and possibly after evaporation of the solvent components—is covered with a further film which will later constitute the backing layer of TDDS. The pharmaceutically acceptable substance suitable as auxiliaries such as plasticizer, tackifiers, solubilizers, stabilizers, fillers, carrier substances and permeation enhancers are in principle known to these skilled in the art.

The device of the present disclosure can release drug continuously by diffusion process. In this mode, the driving force is the difference in Hydroxychloroquine and/or chloroquine activity between the device reservoir and the skin and underlying tissue. The Hydroxychloroquine and/or chloroquine, which is entirely dissolved or disperse in the carrier and/or vehicle and/or polymer system of present disclosure, permeates through the carrier to the skin. The reservoir or matrix system is in diffusion communication with the skin—which means that it either contacts the skin directly or contacts semipermeable material interposed between the reservoir or matrix system and the skin that provide permeation pathway for the Hydroxychloroquine and/or chloroquine and permeation enhancer to migrate from the reservoir or matrix to the skin. The interposed material may be homogenous, heterogeneous, or be composed of multiplicity of distinct layer.

Suitable base polymers for producing the active substance reservoir or matrix or the pressure sensitive adhesive layer of the TDDS as disclosed herein are polymers based on cellulose and its derivatives (methylcellulose, ethyl cellulose, carboxymethyl cellulose, Hydroxypropyl cellulose, hydroxypropylmethyl cellulose etc.), natural polymers, polysaccharides and its derivatives such as but not limited to (agar, alginic acid and derivatives, cassia tora gum, collagen, gelatin, gellan gum, guar gum, pectin, potassium or sodium carrageenan, tragacanth, xanthan gum, copal, starch, chitosan, resin etc.), synthetic polymers and its derivatives such as without any limitation to carboxy vinyl polymers or carbomers (carbopol 940, carbopol 934, carbopol 971), polyethylene and its co-polymers etc. clays such as silicate etc. polyvinyl alcohol, polyacrylamide, polyvinyl pyrrolidone homopolymer and polyvinyl pyrrolidone copolymers (PVP, Poloxamer), acrylic acid its ester, polyacrylate copolymers, isobutylene, ethylene vinyl acetate copolymers, natural rubbers, synthetic rubbers such as styrene-diene copolymers, styrene-butadiene block copolymers, isoprene block copolymers, acrylonitrile butadiene rubber, butyl rubber or neoprene rubber, as well as pressure sensitive adhesive based on silicone, or “hot-melt adhesive”. The term “hot-melt adhesive” comprises any adhesive which are not liquefied with solvent but by melting at elevated temperature, preferably in the range of from 60-200° C. Suitable as hot-melt adhesive are in particular, mixture of esters of hydrogenated colophony with cellulose derivatives. The mentioned base polymers may also be used in form of suitable mixtures.

On top of the above-mentioned polymers other polymers known to the skilled artisan may also be used as a base polymers for producing polymer vehicle or the matrix or the pressure sensitive adhesive layer, provided they are compatible with Tydroxychloroquine and/or chloroquine. In theory, a variety of polymers, resins and additives known to the art can be taken into consideration for production of TDDS. However, care must be take that these substances, in so far as coming into contact with the skin, are tolerated by the skin, and that the formulation is suitable for delivering Hydroxychloroquine and/or chloroquine.

In another embodiment, the active substance, Hydroxychloroquine and/or chloroquine is in the simplest case dispersed, coarsely, colloidally or molecularly, in a solution or melt of base polymers. In the further TDDS manufacturing techniques, the Hydroxychloroquine and/or chloroquine is in the form of supersaturated solution, nano-emulsion or nano-suspension, amorphous, crystalline, co-crystals, coated with base polymers or solubilize in polymers using hot melt extrusion process.

A preferred embodiment of the invention consists in that the active substance Hydroxychloroquine and/or chloroquine is present in the reservoir of TDDS in dissolved condition; in this case the formulation should, if possible, contain a solubilizer. Selected examples for solubilizers are polysorbate such as but not limited to (polysorbate 20, polysorbate 40, polysorbate 60, polysorbate 80 etc), span such as but not limited to (span 80, span 20 etc), surfactants such as (anionic, cationic, nonionic and amphoteric), propylene glycol monocaprylate type 1, propylene glycol monocaprylate type II, propylene glycol dicaprylate, medium chain triglycerides, propylene glycol monolaurate type 1T, linoleoyl polyoxyl-6 glycerides, Caprylic glyceride, oleoyl-polyoxyl-6-glycerides, lauroyl polyoxyl-6-gylcerides, polyglyceryl-3—diolcate, diethylene glycol monoethyl ether, propylene glycol monolaurate type I etc, cyclodextrins, polyhydric alcohol, especially 1,2-propanediol, butanediol, glycerine, polyethylene glycol (m.w. 200 and higher), Dimethyl Sulfoxide, Dimethyl Isosorbide, tetrahydrofurfiiryl alcohol, diethyl tolumide, monoisopropylidene glycerine and others Solubilizers, surfactants, emulsifying agents, dispersing agents and similar compounds or chemicals known to those skilled in the art can be used either alone or in combination thereof. It has proved to be advantageous for the portion of the solubilizer to be 1 to 99% wt, especially preferred 5 to 75% wt. relative to the overall weight of the Hydroxychloroquine and/or chloroquine reservoir. It is to be taken into consideration that some of the mentioned solubilizers, e.g. Dimethyl Sulfoxide, Dimethyl Isosorbide, diethylene glycol monoethyl ether, can simultaneously act as a permeation enhancing agents.

In another embodiment, solvents can be also used to make up the weight of the total reservoir or matrix or pressure sensitive adhesive matrix systems. Theses solvents can also be used to increase the solubility of Hydroxychloroquine and/or chloroquine in the reservoir or matrix systems. Such solvents known to those skilled in the art can be used either alone or in mixture thereof without any limitation to following like alcohol C₁-C₂₀ such as but not limited to (methanol, ethanol, isopropyl alcohol, butanol, propanol etc.), polyhydric alcohols, isopropyl myristate, water, glycols such as but not limited (propylene glycol, polyethylene glycol, dipropylene glycol, hexylene glycol, glycerine etc.), pyrrolidone such as but not limited to (N-methyl 2-pyrrolidone, 2-pyrrolidone etc.), sulfoxides such as but not limited to (dimethyl Sulfoxide, decylmethylsulfoxide etc.), dimethyl Isosorbide, mineral oils, vegetable oils, volatile chemicals which can be used to make matrix patch such as but not limited to (ethanol, propanol, ethyl acetate, acetone, methanol, dichloromethane, chloroform, toluene, Isopropyl alcohol), acids such as but not limited to lactic acid, acetic acid, bases and others.

To achieve a high Hydroxychloroquine and/or chloroquine flux through the skin, it has proved particularly beneficial, especially in matrix or adhesive systems, for the Hydroxychloroquine and/or chloroquine reservoir or matrix or pressure sensitive adhesive to contain permeation enhancing excipients in an amount of 0.1 to 25% wt, preferably from 1 to 15% wt, in each case relative to the total weight of the Hydroxychloroquine and/or chloroquine reservoir or matrix or pressure sensitive adhesive. Preferred example for skin permeation-enhancing agents are water, sulfoxides, and similar chemicals such as but not limited to (dimethylsulfoxide, dimethylacetamide, dimethylformamide, decylmethylsulfoxide, dimethylisosorbide etc), azone, pyrrolidones such as but not limited to (N-methyl-2-pyrrolidone, 2-pyrrolidone etc), esters such as but not limited to (Propylene glycol monolaurate, butyl ethanoate, ethyl ethanoate, isopropyl myristate, isopropyl palmitate, methyl ethanoate, decyl oleate, glycerol monooleate, glycerol monolaurate, lauryl laurate etc), fatty acids such as but not limited to (capric acid, caprylic acid, lauric acid, oleic acid, myristic acid, linoleic acid, stearic acid, palmitic acid etc), alcohols, fatty alcohols and glycols such as but not limited to (oleyl alcohol, ethanol, dodecanol, propylene glycol, glycerol etc), ethers such as but not limited to (diethylene glycol monoethyl ether), urea, polyoxyethylene fatty alcohol ethers, polyoxyethylene fatty acid esters, esters of fatty alcohols, esters of long chain fatty acids with methyl, ethyl or isopropyl alcohol, esters of fatty alcohols with acetic acid, lactic acid, as well as oleic acid diethanolamine, essential oils, terpene and terpenoids such as but not limited to (terpineol, limonene, thymol, eineole etc), surfactant type enhancers (polysorbate 80, polysorbate 20 etc.), liposomes, niosomes, transferomes, ethanosomes, etc and all penetration or permeation enhancers referred in the book “Percutaneous Penetration Enhancers” (Eric W. Smith, Howard 1. Mailbach, 2005. Nov, CRC press). The permeation-enhancing substances mentioned above may be added either singly or as a mixture.

To achieve maximum release at constant release rate, the Hydroxychloroquine and/or chloroquine concentration in the active substance matrix or reservoir or pressure sensitive adhesive is preferably as optimized as possible. In this content, it has to be kept in mind, however, that the physical stability of the active substance may be adversely affected if the concentration is to high due to super-saturation, which causes precipitation. In the current invention of TDDS, the Hydroxychloroquine and/or chloroquine concentrations employed are in the range 0.1 to 50%-wt, in particular from 1 to 25% wt, in each case relative to the total weight of the active substance reservoir or matrix or pressure sensitive adhesives.

Moreover, the Hydroxychloroquine and/or chloroquine matrix or pressure sensitive adhesive or individual layers of the matrix may contain plasticizers which are known to those skilled in the art either alone or in combination thereof without any limitation to following like glycerol and its esters, phosphate esters, glycol derivatives, sugar alcohols, sebacic acid esters, azelate, citric acid esters, tartaric acid esters, adipate, phthalic acid esters, triacetin, oleic acid esters and all the plasticizers which can be used in transdermal drug delivery system referred in the book “Handbook of Plasticizers” (George Wypych, 2004, Chem Tee Publishing). The concentration of these plasticizers may be up to 50% wt. and is preferably between 5 to 25% wt, in each case relative to the active substance matrix total weight.

The system as disclosed herein may also comprise such embodiments where the Hydroxychloroquine and/or chloroquine matrix has a two or multi-layered structure, also called multi-laminate drug in adhesive patch. For example, the various matrix layers may contain polymer constitutes from the above-mentioned polymers. In this case, the matrix layers are differing from each other's in the term of polymer or pressure sensitive or hot melt polymers composition, Hydroxychloroquine and/or chloroquine concentration, different permeating enhancers or solublizers. The layers can be separated using semi-permeable membrane between two distinct drug-in-adhesive layers or multiple drug-in-adhesive layers under a single backing film.

The disclosure provides a transdermal drug delivery system (TDDS) for administration of Hydroxychloroquine and/or chloroquine comprising: an active substance area or reservoir comprises a pharmaceutical composition comprising Hydroxychloroquine and/or chloroquine and at least one excipient; an impermeable backing layer:
optionally, a releasing membrane, which is covered by a detachable backing layer. The disclosure provides a TDDS wherein the active substance area or reservoir is configured as a polymer matrix system, a liquid system, a gel system, or a pressure sensitive adhesive system. The disclosure provides a TDDS, wherein the active substance reservoir is constructed in a pouch-shaped system. The disclosure provides a TDDS, wherein the active substance reservoir is a preparation selected from the group consisting of flowable, viscous, semi-solid, gel-like, liquid preparation, solution, suspension, and emulsion. The disclosure provides a TDDS wherein the active substance reservoir is confined on the skin facing side by an active substance permeable membrane and on the opposite side from the skin by an active substance impermeable layer. The disclosure provides a TDDS comprising an active substance permeable membrane which modifies or controls the rate of active substance release. The disclosure provides a TDDS characterized in that the Hydroxychloroquine and/or chloroquine containing area is a single-, double-, or multilayered active substance matrix.

The disclosure provides a TDDS further comprising an adhesive so that it may be applied as a plaster or bandage. The disclosure provides a TDDS wherein the active substance is a matrix selected from the group consisting of a plastic or synthetic resin matrix, a pressure-sensitive adhesive matrix, wherein the basic polymer(s) of this matrix are selected from the group consisting of polymers based on acrylic acid and its esters, isobutylene, ethylene-vinyl acetate copolymers, natural rubbers, synthetic rubbers, styrene-diene copolymers, styrene-butadiene block copolymers, isoprene block copolymers, acrylonitrile-butadiene rubber, butyl rubber and neoprene rubber, pressure sensitive adhesives based on silicone, hot-melt adhesive, mixtures of esters of hydrogenated colophony with cellulose derivatives, and combinations thereof. The disclosure provides a TDDS wherein the active substance reservoir contains a fiber material, a woven fabric or a nonwoven, to which the active substance is adsorbed. The disclosure provides a TDDS can deliver 1-40 mg/day Hydroxychloroquine and/or chloroquine through the skin to the blood in a subject, which can produce up to 2000 ng/ml plasma concentration. The disclosure provides a TDDS wherein the Hydroxychloroquine and/or chloroquine is present in a concentration in the range of from 0.1-50 wt %, preferably from 1-30 wt %, more preferably 1-20 wt %, in each case relative total mass of the active substance reservoir.

The disclosure provides a TDDS wherein Hydroxychloroquine and/or chloroquine is present in the active substance reservoir either in dissolved or suspended state. The disclosure provides a TDDS wherein the active substance reservoir contains at least one solubilizer, preferably in an amount of from 1 to 99 wt %, with particular preference from 5 to 70 wt %, in each case relative to the total weight of the active substance reservoir. The disclosure provides a TDDS wherein the solubilizer is selected from the group consisting of polysorbate, span, surfactants (anionic, cationic, nonionic and amphoteric), propylene glycol monocaprylate and its derivatives, glycols and its derivatives, triglycerides and its derivatives, diethylene glycol monoethyl ether, cyclodextrins, polyhydric alcohol, polyethylene glycol (m.w. 200 and higher), tetrahydrofurfuryl alcohol, diethyl tolumide, monoisopropylidene glycerine, sulfoxides, and similar chemicals such as but not limited to dimethylsulfoxide, dimethylacetamide, dimethylformamide, decylmethylsulfoxide, dimethylisosorbide, Caprylocaproyl polyoxyl-8 glycerides, triacetine, and combinations thereof.

The disclosure provides a TDDS wherein the active substance reservoir contains at least one permeation-enhancing agent, in an amount of from 0.1 to 50 wt %, with particular reference from 1 to 25 wt %, in each case relative to the total weight of the active substance reservoir. The disclosure provides a TDDS where in the permeation-enhancing agent is selected from the group consisting of azone, pyrrolidones, N-methyl-2-pyrrolidone, 2-pyrrolidone, esters, Propylene glycol monolaurate, butyl ethanoate, ethyl ethanoate, isopropyl myristate, isopropyl palmitate, methyl ethanoate, decyl oleate, glycerol monooleate, glycerol monolaurate, lauryl laurate, fatty acids, alcohols, fatty alcohols and glycols, ethers, urea, polyoxyethylene fatty alcohol ethers, polyoxyethylene fatty acid esters, esters of fatty alcohols, esters of long chain fatty acids with methyl, ethyl or isopropyl alcohol, esters of fatty alcohols, acetic acid, lactic acid, diethanolamine, essential oils, propylene glycol monolaurate type 1 and type II, terpene and terpenoids, surfactant type enhancers, and combinations thereof.

The disclosure provides a TDDS characterized in that during application period of the TDDS an irritation score should be minimum 0 and maximum 2. The disclosure provides a TDDS use of a Hydroxychloroquine and/or chloroquine-containing TDDS according as disclosed herein for treating rheumatoid arthritis, and/or malaria, and/or SARS CoC-2 infection, and/or lupus erythematosus, and/or porphyria cutanea tarda.

The disclosure provides a method of treating and/or preventing rheumatoid arthritis comprising: selecting a patient in need of such treatment and/or prevention; applying to the skin of the patient a TDDS of the invention; thereby treating and/or preventing the rheumatoid arthritis. The disclosure provides a method of treating and/or preventing lupus erythematosus comprising: selecting a patient in need of such treatment and/or prevention; applying to the skin of the patient a TDDS of the invention. The disclosure provides a method of treating and/or preventing malaria comprising: selecting a patient in need of such treatment and/or prevention; applying to the skin of the patient a TDDS of the invention. The disclosure provides a method of treating and/or preventing SARS CoV-2 infection comprising: selecting a patient in need of such treatment and/or prevention; applying to the skin of the patient a 1DDS of the invention. The disclosure provides a method of treating and/or preventing prophyra cutanea tarda comprising: selecting a patient in need of such treatment and/or prevention; applying to the skin of the patient a TDDS as disclosed herein.

The disclosure provides a method according characterized in that the application period of the TDDS is at least 24 hr and maximally 7 days.

The disclosure provides a method of making a transdermal drug delivery system (TDDS) for administration of Hydroxychloroquine and/or chloroquine comprising: providing an active substance area or reservoir;

providing an impermeable backing layer; optionally providing a releasing membrane, which is covered by a detachable backing layer, wherein the active substance area or reservoir comprises a pharmaceutical composition comprising Hydroxychloroquine and/or chloroquine and at least one excipient.

The disclosure provides a Hydroxychloroquine and/or chloroquine-containing TDDS for use in the preparation of a medicament for use in treating and/or preventing rheumatoid arthritis or treating and/or preventing malaria or treating and/or preventing lupus erythematosus or treating and/or preventing SARS CoV-2 infection or treating and/or preventing porphyria cutanea tarda. A method for treating or preventing a disease or condition in a patient, wherein the disease or condition is selected from the group consisting of rheumatoid arthritis and/or lupus erythematosus and/or malaria and/or SARS CoV-2 infection and/or porphyria cutanea tarda, and combinations thereof, wherein said method comprises: selecting a patient in need of treating or preventing said disease or condition; administering to the patient the composition of the invention in a therapeutically effective amount, thereby treating or preventing said disease in said patient.

### DETAILED DESCRIPTION OF THE INVENTION

Hydroxychloroquine and/or chloroquine refers to all pharmaceutically acceptable forms of Hydroxychloroquine and/or chloroquine either alone or in combinations thereof, for example in following forms but not limited to such as free base or salt or isomer or amorphous or crystalline or co crystalline or solid solution or prodrug or analog or derivatives or metabolites.

RA: Rheumatoid Arthritis

SARS CoV: Severe acute Respiratory Coronavirus

LE: Lupus Erythematosus

PCT: Prophyria Cutanea Tarda

TDDS: Transdermal drug delivery system

TDS: Transdermal delivery system

### Range:

Terms transdermal and topical are used interchangeably.

Terms Formulation and composition are used interchangeable.

Terms Transdermal drug delivery system and transdermal delivery system are used Interchangeably.

Terms reservoir system and reservoir patch are used interchangeably.

Terms matrix system and matrix patch are used interchangeably.

Terms transdermal composition and pharmaceutical composition are used interchangeably.

Term liquid includes without any limitation solution, suspension, micro suspension, nano suspension, dispersion, sprays, aerosols, where solutions are preferred.

The term semisolid includes without any limitation such as gels, ointments, creams, emulsion, microemulsion, nanoemulsion, paste, balms, magma, lotions, mousses, waxes, where gels are preferred.

The term polymer film includes polymer without any limitation pressure sensitive adhesive and/or non-adhesive polymer.

Transdermal delivery system: Reservoir system and/or matrix system comprising Pharmaceutical composition.

All the pharmaceutical compositions are percent by weight.

Without any limitation enhancers used in liquid formulation can be used for semi solid and polymer formulation.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

As used herein, the term “agent” refers to any molecule, compound, methodology and/or substance for use in the prevention, treatment, management and/or diagnosis of a disease or condition. As used herein, the term “effective amount” refers to the amount of a therapy that is sufficient to result in the prevention of the development, recurrence, or onset of a disease or condition, and one or more symptoms thereof, to enhance or improve the prophylactic effect(s) of another therapy, reduce the severity, the duration of a disease or condition, ameliorate one or more symptoms of a disease or condition, prevent the advancement of a disease or condition, cause regression of a disease or condition, and/or enhance or improve the therapeutic effect(s) of another therapy.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the term “therapeutic agent” refers to any molecule, compound, and/or substance that is used for the purpose of treating and/or managing a disease or disorder.

As used herein, the terms “therapies” and “therapy” can refer to any method(s), composition(s), and/or agent(s) that can be used in the prevention, treatment and/or management of a disease or condition, or one or more symptoms thereof. In certain embodiments, the terms “therapy” and “therapies” refer to small molecule therapy.

As used herein, the terms “treat,” “treatment,” and “treating” in the context of the administration of a therapy to a subject refer to the reduction or inhibition of the progression and/or duration of a disease or condition, the reduction or amelioration of the severity of a disease or condition, such as cancer, and/or the amelioration of one or more symptoms thereof resulting from the administration of one or more therapies.

The term “derivative” or “derivatized” as used herein includes chemical modification of a compound of the invention, or pharmaceutically acceptable salts thereof or mixtures thereof. That is, a “derivative” may be a functional equivalent of a compound of the invention, which is capable of inducing the improved pharmacological functional activity in a given subject. Illustrative of such chemical modifications would be replacement of hydrogen by a halo group, an alkyl group, an acyl group or an amino group.

As used herein, the term “pharmaceutically acceptable salts” includes acid addition salts or addition salts of free bases. The term “pharmaceutically acceptable salts” of a compound of the invention is also meant to include within its scope all the possible isomers and their mixtures, and any pharmaceutically acceptable metabolite, bioprecursor and/or pro-drug, such as, for example, a compound which has a structural formula different from the one of the compounds of the invention, and yet is directly or indirectly converted in vivo into a compound of the invention, upon administration to a subject, such as a mammal, particularly a human being.

The compound may be in the form of a pharmaceutically acceptable salt, such as an acid addition salt or a base salt, or a solvate thereof, including a hydrate thereof. Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartaric, citrate, gluconate, succinate, saccharate, benzoate, methanesulphonate, ethanesulphonate, benzcnesulphonate, p-toluenesulphonate and pamoate salts. Suitable base salts are formed from bases which form non-toxic salts and examples are the sodium, potassium, aluminium, calcium, magnesium, zinc and diethanolamine salts.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein, the term “transdermal” refers to delivery, administration or application of a drug by means of direct contact with skin or mucosa. Such delivery, administration or application is also known as dermal, percutaneous, transmucosal and buccal. As used herein, “dermal” includes skin and mucosa, which includes oral, buccal, nasal, rectal and vaginal mucosa.

As used herein, “transdermal drug delivery system” refers to a system (e.g., a device) comprising a composition that releases drug upon application to the skin (or any other surface noted above). A transdermal drug delivery system may comprise a drug-containing composition, and, optionally, a backing layer and/or a release liner layer. In some embodiments, the transdermal drug delivery system is a substantially non-aqueous, solid form, capable of conforming to the surface with which it comes into contact, and capable of maintaining such contact so as to facilitate topical application without adverse physiological response, and without being appreciably decomposed by aqueous contact during topical application to a subject. Many such systems are known in the art and commercially available, such as transdermal drug delivery patches. Typically, transdermal drug delivery systems are classified into one of two categories: matrix-type systems and reservoir-type systems, as discussed in more detail below.

A transdermal drug delivery system also may include a drug impermeable backing layer or film. In some embodiments, the backing layer is adjacent the drug-containing composition. When present, the backing layer protects the polymer matrix layer (and any other layers present) from the environment and prevents loss of the drug and/or release of other components to the environment during use. Materials suitable for use as backing layers are well-known known in the art and can comprise films of polyester, polyethylene, vinyl acetate resins, ethylene/vinyl acetate copolymers, polyvinyl chloride, polyurethane, and the like, metal foils, non-woven fabric, cloth and commercially available laminates. A typical backing material has a thickness in the range of 2 to 1000 micrometers. For example, 3M's Scotch Pak® 1012 or 9732 (a polyester film with an ethylene vinyl acetate copolymer heat seal layer), 9723 (a laminate of polyethylene and polyester), or CoTran 9720 (a polyethylene film) are useful in the transdermal drug delivery systems described herein, as are Dow® backing layer films, such as Dow® BLF 2050 (a multi-layer backing comprising ethylene vinyl acetate layers and an internal SARAN® layer.

A transdermal drug delivery system also may include a release liner, typically located adjacent the opposite face of the system as compared to the backing layer. When present, the release liner is removed from the system prior to use to expose the polymer matrix layer and/or an adhesive layer prior to topical application. Materials suitable for use as release liners are well-known known in the art and include the commercially available products of Dow Corning Corporation designated Bio-Release® liner and Syl-off® 7610, Loparex's PET release liner (silicone-coated) and 3M's 1020, 1022, 9741, 9744, 9748, 9749 and 9755 Scotchpak.™. (fluoropolymer-coated polyester films).

A transdermal drug delivery system may be packaged or provided in a package, such as a pouchstock material used in the prior art for transdermal drug delivery systems in general. For example, DuPont's Surlyn® can be used in a pouchstock material. Alternatively, a pouchstock comprising a coextruded ethylene acrylic acid/low-density polyethylene (EAA/LDPE) material, or Barex® from INEOS (acrylonitrile-methyl acrylate) may be used.

The disclosure provides pharmaceutical composition for transdermal delivery of Hydroxychloroquine and/or chloroquine up to 7 days.

In one embodiment, the disclosure provides pharmaceutical compositions as liquid formulation for transdermal delivery of Hydroxychloroquine and/or chloroquine. In one aspect the invention further provides liquid formulation comprising Hydroxychloroquine and/or chloroquine and vehicle system. The disclosure further provides the vehicle system, which comprises solvents (solubilizer), permeation enhancing agents, if required acid or base for pH adjustment mentioned should be use. The liquid formulation comprising Hydroxychloroquine and/or chloroquine and vehicle system is preferred.

In one aspect liquid formulation comprise Hydroxychloroquine and/or chloroquine and vehicle system wherein, Hydroxychloroquine and/or chloroquine is present in an amount between 0.1-50 wt %, vehicle system is present in an amount between 5-99.9 wt %. More preferably, Hydroxychloroquine and/or chloroquine is present in an amount between 1-25 wt %, vehicle system is present in an amount between 1-99 wt %. The invention further provides an exemplary composition of the invention comprising about 0.1-50 wt % Hydroxychloroquine and/or chloroquine, 0.1-99.9 wt % dimethylsulfoxide, 0.1-99.9 wt % dimethylisosorbide, 0.1-99.9 wt % dipropylene glycol, 0.1-99.9 wt % highly purified diethylene glycol monoethyl ether, 0.1-50 wt % fatty acid, 0.1-50 wt % Lactic acid, 0.1-99.9% wt propylene glycol, 0.1-99.9% wt polyethylene glycol-⁴00, 0.1-50% wt water, pH between 3.5-8. More Preferably, about 1-25 wt % Hydroxychloroquine and/or chloroquine, 5-50 wt % dimethylsulfoxide, 5-50 wt % dimethylisosorbide, 1-25 wt % dipropylene glycol, 1-50 wt % highly purified diethylene glycol monoethyl ether, 0.1-20 wt %, fatty acid, 0.1-20 wt % Lactic acid, 1-25% wt propylene glycol, 1-25% wt polyethylene glycol-400, 1-25% wt water, pH adjusted between 4-7. Without limiting in scope an exemplary formulation in this range is illustrated in Example 1.

In another embodiment, The disclosure provides pharmaceutical compositions as semisolid formulation for transdermal delivery of Hydroxychloroquine and/or chloroquine for up to 7 days.

In one aspect the disclosure further provides semisolid formulation comprising Hydroxychloroquine and/or chloroquine and polymeric vehicle system. The invention further provides the vehicle system, which comprise solvents (Solubilizer), permeability enhancing excipients and polymer or gelling agent or thickening agent, if required acid or base for pH adjustment. The semisolid formulation comprising Hydroxychloroquine and/or chloroquine and a polymeric vehicle system is preferred.

One aspect of semisolid formulation comprises Hydroxychloroquine and/or chloroquine and a polymeric vehicle system wherein, Hydroxychloroquine and/or chloroquine is present in an amount between 0.1-50 wt %, and the polymeric vehicle system is present in an amount between 0.1-99.9 wt %. More preferably, Hydroxychloroquine and/or chloroquine is present in an amount between 1-30 wt/o, and the polymeric vehicle system is present in an amount between 25-99 wt % to make up to 100 wt %

The disclosure further provides an exemplary formulation of the invention comprising about 0.1-50 wt % Hydroxychloroquine and/or chloroquine, 0.5-99.9 wt % dimethylsulfoxide, 0.5-99.9 wt % polyethylene glycol-400, 0.5-99.9 wt % diethylene glycol monoethyl ether, 0.5-99.9% wt propylene glycol, 0.5-99.9 wt % dipropylene glycol, 0.1-50 wt % Lactic acid, 0.5-99.9 wt %, dimethyl isosorbide, 0.5-50 wt % fatty acid, 0,5-50% wt water, 0.1-50% wt polyvinyl pyrrolidone, pH between 3.5-8. More Preferably, about 0.1-25 wt % Hydroxychloroquine and/or chloroquine, 0.5-50 wt % dimethylsulfoxide, 0.5-50 wt % polyethylene glycol-400, 0.5-50 wt % diethylene glycol monoethyl ether, 0.5-50% wt propylene glycol, 0.5-50 wt % dipropylene glycol, 0.1-20 wt % Lactic acid. 0.5-50 wt %, dimethyl isosorbide, 0.5-50 wt % fatty acid, 0.5-50% wt water, 0.1-30% wt polyvinyl pyrrolidone, pH adjusted between 4-7.

The disclosure further provides another exemplary formulation of the invention comprising about 0.1-25 wt % Hydroxychloroquine and/or chloroquine, 0.5-50 wt % dimethylsulfoxide, 0.5-50 wt % polyethylene glycol-400, 0.5-50 wt % highly purified diethylene glycol monoethyl ether, 0.5-50% wt propylene glycol, 0.5-50 wt % dipropylene glycol, 0.1-20 wt % Lactic acid, 0.5-50 wt %, dimethyl isosorbide, 0.5-50 wt % fatty acid, 0.5-50% wt water, 0.1-30% wt polyvinyl pyrrolidone, 0.1-15 wt % hydroxypropyl cellulose HF, pH adjusted between 4-7.

The disclosure further provides yet another exemplary formulation of the invention comprising about 0.1-25 wt % Hydroxychloroquine and/or chloroquine, 0.5-50 wt % dimethylsulfoxide, 0.5-50 wt % polyethylene glycol-400, 0.5-50 wt % highly purified diethylene glycol monoethyl ether, 0.5-50% wt propylene glycol, 0.5-50 wt % dipropylene glycol, 0.1-20 wt % Lactic acid, 0.5-50 wt %, dimethyl isosorbide, 0.5-30 wt % Caprylocaproyl polyoxyl-8 glycerides, 0.5-50 wt % Propylene glycol monolaurate type II, 0.5-30% wt Tween-20, 0.1-15 wt % hydroxypropyl cellulose HF, pH adjusted between 4-7. Without limiting in scope exemplary formulations in this range is illustrated in examples.

The disclosure further provides yet another exemplary formulation of the invention comprising about 0.1-25 wt % Hydroxychloroquine and/or chloroquine, 0.5-50 wt % dimethylsulfoxide, 0.5-50 wt % Hexylene Glycol, 0.5-50 wt % highly purified diethylene glycol monoethyl ether, 0.5-50 wt % Triacetine, 0.1-20 wt % Lactic acid, 0.5-50 wt %, dimethyl isosorbide, 0.5-30 wt % Caprylocaproyl polyoxyl-8 glycerides, 0.5-50 wt % fatty acid, 0.5-50% wt water, 0.1-30% wt polyvinyl pyrrolidone, 0.1-15 wt % hydroxypropyl cellulose HF, pH adjusted between 4-7.

The disclosure further provides yet another exemplary formulation of the invention comprising about 0.1-25 wt/o Hydroxychloroquine and/or chloroquine, 0.5-50 wt % dimethylsulfoxide, 0.5-50 wt % Hexylene Glycol, 0.5-50 wt % highly purified diethylene glycol monoethyl ether, 0.5-50 wt % Triacetine, 0.1-20 wt % Lactic acid, 0.5-50 wt %, dimethyl isosorbide, 0,5-30 wt/o Caprylocaproyl polyoxyl-8 glycerides, 0.5-50 wt % fatty acid, 0.5-50% wt water, 0.1-30% wt polyvinyl pyrrolidone, 0.1-15 wt % hydroxypropyl cellulose HF, pH adjusted between 4-7.

The disclosure further provides yet another exemplary formulation of the invention comprising about 0.1-25 wt % Hydroxychloroquine and/or chloroquine, 0.5-99 wt % dimethylsulfoxide, 0.5-99 wt % polyethelene glycol-400, 0.5-99% wt propylene glycol, 0.5-99 wt % Propylene glycol monolaurate type II, 0.5-50% wt water, 0.1-15 wt % hydroxypropyl cellulose HF, pH adjusted between 4-7. Without limiting in scope exemplary formulations in this range is illustrated in examples.

The disclosure pertains to the transdermal delivery of Hydroxychloroquine and/or chloroquine for the treatment of RA and/or Malaria and/or LE and/or PCT and/or SARS CoV. Another embodiment pertains to the use of acrylic or silicone pressure sensitive adhesive and/or polymer matrix which do not contain functional groups and which are not cross linked, but are able to absorb or solubilize large amount of Hydroxychloroquine and/or chloroquine and at the same time provide equal or better adhesion to skin and permeation through human skin. More preferred examples of pressure sensitive adhesive (PSAs), that could be used but not limited to, include those based on pure acrylate monomers as well as acrylate copolymers and terpolymers using for example as the co-monomers vinyl acetate or hydrocarbon copolymers which may also include pacifiers and other pressure sensitive adhesive modifiers. Some examples of these PSAs are Durotak 87-900A, 87-901A, 87-2516, 87-9301, Bio PSA-4202, Bio-PSA 4302, Bio-PSA 4502, Bio PSA-4602 and etc.

Another embodiment of disclosure is to inhibit crystallization in matrix patches using solubilizer an/or solvents and/or permeability enhancing agents by providing stabilization of the patch through absorption and immobilization of the liquid in the patch. For example of such excipients include but not limited to PVP, PVP/PVA, hydroxypropylcellulose, hydroxyethylcellulose, methyl cellulose, sodium carboxymethyl cellulose, colloidal silica, Xanthan gum, and etc.

Another embodiment is in matrix and/or drug-in-adhesive and/or drug-in-polymer with two kinds of enhancers, volatile and non-volatile. Volatile enhancers are the excipients that have a vapor pressure 0.2 mmHg and higher at 20° C. such as dimethylsulfoxide, dimethylisosorbide, diethylene glycol monoethyl ether and etc., while, the non-volatile enhancers are the liquids that have a vapor pressure less than 0.2 mm Hg at 20° C. such as urea, lauryl lactate and etc. Volatile enhancers are the enhancers that will evaporate during drying process of matrix and/or drug-in-adhesive and/or drug-in-polymer preparation.

In another embodiment of the disclosure is a formulation comprising about 0.1-99 wt % Hydroxychloroquine and/or chloroquine, 0.5-99 wt % dimethylsulfoxide. 0.5-99 wt % Triacetine, 0.5-99 wt % highly purified diethylene glycol monoethyl ether, 0.5-99 wt % propylene glycol monoluarate type II, 0.1-99 wt % adhesive. More preferably 0.1-50 wt % Hydroxychloroquine and/or chloroquine, 0.5-50 wt % dimethylsulfoxide, 0.5-50 wt % Triacetine, 0.5-50 wt % highly purified diethylene glycol monoethyl ether, 0.5-50 wt % propylene glycol monoluarate type II, 0.1-90 wt % adhesive.

The invention will be illustrated in more detail with reference to the following Examples, but it should be understood that the present invention is not deemed to be limited thereto.

### EXAMPLES

### Example 1

This Example describes the preparation of a patch or semisolid formulation, which must give a blood level (±20%) bioequivalent to 50 mg oral Hydroxychloroquine and/or chloroquine. Initially, a transdermal formulation will be prepared containing a dose of 50 mg Hydroxychloroquine based on the in-vitro permeability flux profile obtained from Franz-diffusion cells, the dose will be adjusted to obtain desired blood level (±20%) bioequivalent to oral 50 mg/day Hydroxychloroquine and/or chloroquine. Different approaches will be implemented (e.g. change in drug loading dose, combination of solvents/enhancers etc.) to prepare a transdermal formulation which can deliver target therapeutic blood level from day 1 to day 5 or day 7.

### Example 2

Below is a description of the experimental procedure, utilized for development and optimization of transdermal matrix patch or transdermal semisolid formulation containing Hydroxychloroquine and/or chloroquine, or a combination of hydroxychloroquine and/or chloroquine. Exemplary formulations are set forth in Table 1:

TABLE 1

| | HCQ 1 | HCQ 2 | HCQ 3 | HCQ 4 |
|---|---|---|---|---|
| Excipients | (% w/w) | (% w/w) | (% w/w) | (% w/w) |
| Hydroxy- | 0.1-20% | 0.1-20% | 0.1-20% | 0.1-20% |
| chloroquine/ | | | | |
| Chloroquine | | | | |
| Enhancers | | 0.1-20% | | 0.1-20% |
| Solvents | | | 0.1-20% | 0.1-20% |
| Adhesive/ | 80-99.9% | 50-99.8% | 50-99.8% | 30-99.7% |
| Polymers | | | | |

The transdermal formulation and/or topical formulation of the disclosure may comprise solvents known to those skilled in the art either alone or in combinations thereof without any limitation to following like alcohol C₁-C₂₀ such as but not limited to (methanol, ethanol, isopropyl alcohol, butanol, propanol etc.), polyhydric alcohols, glycols such as but not limited to (propylene glycol, polyethylene glycol, dipropylene glycol, hexylene glycol, butyene glycol, glycerine etc.), derivative of glycols, pyrrolidone such as but not limited to (N methyl 2—pyrrolidone, 2-pyrrolidone etc.), sulfoxides such as but not limited to (dimethyl sulfoxide, decylmethylsulfoxide etc), dimethylisosorbide, mineral oils, vegetable oils, sesame oil water, polar solvents, semi polar solvents, non polar solvents, volatile chemicals which can be used to make matrix patch such as but not limited to (ethanol, propanol, ethyl acetate, acetone, methanol, dichloromethane, chloroform, toluene, IPA, hexane), acids such as but not limited to acetic acid, lactic acid, levulinic acid, bases and others, pentane, dimethylformamide, butane, lipids. More preferably in the range of 0.01%-95% w/w or w/v. In exemplary embodiments, formulations of the disclosure may comprise solvents at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80%, and about 95% of the formulation. In exemplary embodiments, formulations of the disclosure may comprise solvents at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the solvents will represent approximately 1 wt % to 75 wt %, preferably 2 wt % to 30 wt %, more preferably 5 wt. % to 20 wt. % of the formulation.

The transdermal formulation and/or topical formulation of the disclosure may comprise gelling agents and/or thickening and/or suspending agents and/or polymers and/or adhesive polymers and/or pressure sensitive adhesive polymers known to those skilled in the art either alone or in combinations thereof without any limitation to following like natural polymers, polysaccharides and its derivatives such as but not limited to (agar, alginic acid and derivatives. cassia tora, collagen, gelatin, gellum gum, guar gum, pectin, potassium, or sodium carageenan, tragacanth, xantham, gum copal, chitosan, resin etc.), semisynthetic polymers and its derivatives such as without any limitation to cellulose and its derivatives (methylcellulose, ethyl cellulose, carboxymethyl cellulose, hydroxylpropyl cellulose, hydroxypropylmethyl cellulose etc.), synthetic polymers and its derivatives such as without any limitation to carboxyvinyl polymers or carbomers (carbopol 940, carbopol 934, carbopol 971p NF), polyethylene, and its copolymers etc, clays such as but not limited to (silicates, bentonite), silicon dioxide, polyvinyl alcohol, acrylic polymers (eudragit), acrylic acid esters, polyacrylate copolymers, polyacrylamide, polyvinyl pyrrolidone homopolymer and polyvinyl pyrrolidone copolymers such as but not limited to (PVP, Kollidon 30, poloxamer), isobutylene, ethyl vinyl acetate copolymers, natural rubber, synthetic rubber, pressure sensitive adhesives such as silicone polymers such as but not limited to (bio psa 4302, bio-psa 4202 etc.,), acrylic pressure sensitive adhesives such as but not limited to (duro-tak 87-2156, duro-tak 387-2287, duro-tak 87-9301, duro-tak 387-2051 etc.), polyisobutylene such as but not limited to (polyisobutylene low molecular weight, plyisobutylene medium molecular weight, polyisobutylene 35000 mw, etc), acrylic copolymers, rubber based adhesives, hot melt adhesives, styrene-butadiene copolymers, bentonite, all water and/or organic solvent swellable polymers, etc. In exemplary embodiments, formulations of the disclosure may comprise gelling agents and/or thickening and/or suspending agents and/or polymers and/or adhesive polymers and/or pressure sensitive adhesive polymers at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80%, and about 85%, and about 90% of the formulation. In exemplary embodiments, formulations of the disclosure may comprise gelling agents and/or thickening and/or suspending agents and/or polymers and/or adhesive polymers and/or pressure sensitive adhesive polymers at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the gelling agents and/or thickening and/or suspending agents and/or polymers and/or adhesive polymer and/or pressure sensitive adhesive polymers will represent approximately 1 wt % to 75 wt %, preferably 2 wt % to 30 wt %, more preferably 5 wt. % to 20 wt. % of the formulation, and more preferably in the range of 0.1% 80% w/w or w/v.

The transdermal formulation and/or topical formulation of the disclosure may comprise permeation enhancers known to those skilled in the art either alone or in combination thereof without any limitation to the following, such as sulfoxides, and similar chemicals such as but not limited to (dimethylsulfoxide, dimethylacetamide, dimethylformamide, decylmethylsulfoxide, dimethylisosorbide etc), azone, pyrrolidones such as but not limited to (N-methyl-2-pyrrolidone, 2-pyrrolidon etc.), esters, fatty acid esters such as but not limited to (propylene glycol monolaurate, butyl ethanoate, ethyl ethanoate, isopropyl myristate, isopropyl palmitate, methyl ethanoate, lauryl lactate, ethyl oleate decyl oleate, glycerol monooleate, glycerol monolaurate, lauryl laurate etc.), fatty acids such as but not limited to (capric acid, caprylic acid, lauric acid, oleic acid, myristic acid, linoleic acid, stearic acid, palmitic acid etc.), alcohols, fatty alcohols and glycols such as but not limited to (oleyl alcohol, nathanol, dodecanol, propylene glycol, glycerol etc.), ethers alcohol such as but not limited to (diethylene glycol monoethyl ether), urea, triglycerides such as but not limited to triacetin, polyoxyethylene fatty alcohol ethers, polyoxyethylene fatty acid esters, esters of fatty alcohols, essential oils, surfactant type enhancers such as but not limited to (brij, sodium lauryl sulfate, tween, polysorbate), terpene, terpenoids and all penetration or permeation enhancers referred in the book “Percutaneous Penetration Enhancers” (Eric W. Smith, Howard I. Maibach, 2005. Nov. CRC press). In exemplary embodiments, formulations of the disclosure may comprise permeation enhancers at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise permeation enhancers at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the permeation enhancers will represent approximately 1 wt % to 75 wt %, preferably 2 wt % to 30 wt %, more preferably 5 wt. % to 20 wt. % of the formulation, and more preferably in the range of 0.01%-95% w/w or w/v.

All of the components from Table 1, with the exception of the Hydroxychloroquine and/or chloroquine, were mixed together with stirring for 18 hours. Next, the H y droxychloroquine and/or chloroquine was added into the excipient mixture to prepare the final transdermal formulations.

### Example 3

The following steps are provided using composition HCQ and/or CQ 1 as an example for preparing a transdermal patch. The above ingredients are blended by stirring for 18 hours and then, using a commercial benchtop spreader, the matrix is evenly spread onto an 8×14 inch sheet of release liner (such as 3M 9744) to a thickness of 0.5 mm.

The sheet is then placed in an oven at 110° F. for one hour to evaporate off the ethyl acetate adhesive solvent. An opaque backing membrane (such as 3M 9730 NR film) with low permeability to oxygen to inhibit photo and oxidative degradation is then carefully applied by hand to avoid formation of bubbles and voids. A circular die (1.5 inches diameter) is used to cut patches (7 sqcm) for subsequent studies. After drying, the drug adhesive matrix has a surface density of 5-30 mg/sqcm, containing hydroxychloroqyine in 0.1-20% w/w.

### Example 4

The prepared transdermal formulations were then subjected to a flux measurement test as follows. Human cadaver skin, stored at −80° C., was thawed at room temperature in phosphate buffered saline (PBS), and visually inspected for defects before using in the study. Transdermal flux was then measured using standard Franz diffusion cells comprising a cylindrical donor compart and a separate water jacketed cylindrical receptor compartment with the volume of 13 mL The cadaver skin was clamped between the two compartments with the dermis side facing toward the receptor compartment. The donor compartment was filled with the transdermal Hydroxychloroquine and/or chloroquine formulations prepared as described above. The receptor compartment was filled with receptor medium, held at constant temperature, and constantly stirred to collect the Hydroxychloroquine and/or chloroquine as it diffuses through the skin and into receptor compartment. It is important to confirm that the receptor fluid is always in contact with the skin. The receptor compartment was emptied at 24 hr intervals for assay of Hydroxychloroquine and/or chloroquine and replaced with fresh receptor solution. In order to maintain the sink condition in the receptor compartment, it is important to keep the Hydroxychloroquine and/or chloroquine concentration in the receptor compartment less than 10% of its solubility.

The transdermal formulation and/or topical formulation of the disclosure may comprise plasticizers known to those skilled in the art either alone or in combination thereof without any limitation to following like glycerol and its esters, phosphate esters, glycol derivatives, sugar alcohols, sebacic acid esters, citric acid esters, tartaric acid esters, adipate, phthalic acid esters, triacetin, oleic acid esters and all the plasticizers which can be used in transdermal drug delivery system referred in the book “Handbook of Plasticizers” (George Wypych, 2004, Chem Tec Publishing). In exemplary embodiments, formulations of the disclosure may comprise plasticizers at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise plasticizers at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the plasticizers will represent approximately 1 wt % to 75 wt %, preferably 2 wt % to 30 wt %, more preferably 5 wt. % to 20 wt. % of the formulation. More preferably in the range of 0.01%-95% w/w or w/v.

### Example 5

The transdermal formulation and/or topical formulation of the disclosure may comprise emollients, humectants, skin irritation reducing agents and similar compounds or chemicals known to those skilled in the art either alone or in combinations thereof without any limitation to following like petrolatum, lanolin, mineral oil, dimethicone, zinc oxide, glycerin, propylene glycol and others. More preferably in the range of 0.01%-95% w/w or w/v. In exemplary embodiments, formulations of the disclosure may comprise emollients, humectants, skin irritation reducing agents and similar compounds at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise emollients, humectants, skin irritation reducing agents and similar compounds at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the emollients, humectants, skin irritation reducing agents and similar compounds will represent approximately 1 wt % to 75 wt %, preferably 2 wt % to 30 wt %, more preferably 5 wt. % to 20 wt. % of the formulation, and more preferably in the range of 0.01%-95% w/w or w/v.

### Example 6

The transdermal formulation and/or topical formulation of the disclosure may comprise solubilizers, surfactants, emulsifying agents, dispersing agents and similar compounds or chemicals known to those skilled in the art either alone or in combination thereof without any limitation to following like polysorbate such as but not limited to (polysorbate 20, polysorbate 40, polysorbate 60, polysorbate 80 etc.), span such as but not limited to (span 80, span 20 etc.), surfactants such as (anionic, cationic, nonionic and amphoteric), propylene glycol monocaprylate type 1, propylene glycol monocaprylate type II, propylene glycol dicaprylate, medium chain triglycerides, propylene glycol monolaurate type 11, linoleoyl polyoxyl-6 glycerides, oleoyl-polyoxyl-6-glycerides, lauroyl polyoxyl-6-gylcerides, polyglyceryl-3—dioleate, diethylene glycol monoethyl ether, propylene glycol monolaurate type I, polyglyceryl-3-dioleate, caprylocaproyl polyoxyl 8 glycerides etc, cyclodextrins and others. More preferably in the range of 0.01% 95% w/w or w/v. In exemplary embodiments, formulations of the disclosure may comprise solubilizers, surfactants, emulsifying agents, dispersing agents and similar compounds at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise solubilizers, surfactants, emulsifying agents, dispersing agents and similar compounds at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the solubilizers, surfactants, emulsifying agents, dispersing agents and similar compounds will represent approximately 1 wt % to 75 wt %, preferably 2 wt % to 30 wt %, more preferably 5 wt. % to 20 wt. % of the formulation, and more preferably in the range of 0.01% 95% w/w or w/v.

### Example 7

Different techniques and ingredients can be used to increase the stability and/or solubility of the active agents in formulation such as without any limitation to coating, encapsulation, microencapsulation, nanoencapsulation, lyophilization, chelating agents, complexing agents, etc.

### Example 8

The transdermal formulation and/or topical formulation of the disclosure may comprise auxiliary pH buffering agents and pH stabilizers and similar compounds known to those skilled in the art which helps to maintain the appropriate pH of formulation preferably in the range of 4.0-8.0 either alone or in combination thereof without any limitation to following such as phosphate buffer, acetate buffer, citrate buffer, etc., acids such as but not limited to (carboxylic acids, inorganic acids, sulfonic acids, vinylogous carboxylic acids and others), base such as but not limited to (sodium hydroxide, potassium hydroxide, ammonium hydroxide, triethylamine, sodium carbonate, sodium bicarbonate) etc. More preferably in the range of 0.01%-30% w/w or w/v. In exemplary embodiments, formulations of the disclosure may comprise pH buffering agents and pH stabilizers and similar compounds at a concentration of about 0.01%, about 0.02%, about 0.05%, about 0.1%, about 0.2%, about 0.3%, about 0.4%, about 0.5%, about 0.6%, about 0.7%, about 0,8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise pH buffering agents and pH stabilizers and similar compounds at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the pH buffering agents and pH stabilizers and similar compounds will represent approximately 1 wt % to 75 wt %, preferably 2 wt % to 30 wt %, more preferably 5 wt. % to 20 wt. % of the formulation, and more preferably in the range of 0.01%-30% w/w or w/v.

### Example 9

The transdermal formulation and/or topical formulation of the disclosure may comprise antioxidants such as but not limited to (sodium metabisulfite, citric acid, ascorbic acid, B11A, BHT), oxidizing agents, stabilizers, discoloring agents, preservatives and similar compounds or chemicals known to those skilled in the art which helps to get a stable formulation can be used either alone or in combination thereof without any limitation. More preferably in the range of 0.01%-50% w/w or w/v. In exemplary embodiments, formulations of the disclosure may comprise antioxidants at a concentration of about 0.01%, about 0.02%, about 0,05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise antioxidants at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the antioxidants will represent approximately 1 wt % to 75 wt %, preferably 2 wt % to 30 wt %, more preferably 5 wt. % to 20 wt. % of the formulation, and more preferably in the range of 0.01%-50% w/w or w/v.

### Example 10

The transdermal formulation and/or topical formulation of the disclosure may be formulated in ointment and/or cream base and/or gel and/or film forming formulation and/or transdermal matrix formulation and/or drug-in-adhesive matrix patch and/or matrix patch known to those skilled in the art.

### Example 11

Materials to make the transdermal delivery system of the disclosure in patch form known to those skilled in the art, for example, such as but not limited to reservoir patch, matrix patch, drug in adhesives, film forming formulation, micro-dosing transdermal patch, transdermal films and may include, such as but are not limited to polymers, copolymers, derivatives, backing film, release membranes, release liners, etc. either alone or in combinations thereof. Pressure sensitive adhesives (such as but not limited to silicone polymers, rubber based adhesives, acrylic polymers, acrylic copolymers, polyisobutylene, acrylic acid—isooctyl acrylate copolymer, hot melt adhesives, polybutylene etc.), backing film (such as but not limited to ethylene vinyl acetate copolymers, vinyl acetate resins, polyurethane, polyvinyl chloride, metal foils, polyester, aluminized films, polyethylene, etc.), release membrane (such as but not limited to microporous polyethylene membrane, microporous polypropylene membrane, rate controlling ethylene vinyl acetate copolymer membrane etc.), release liners (such as but not limited to siliconized polyester films, fluoropolymer coated polyester film, polyester film, siliconized polyethylene terephthalate film, etc.), tapes, etc.

The transdermal formulation and/or topical formulation and/or transdermal delivery system of the disclosure may deliver at least therapeutic effective dose of active agent, Hydroxychloroquine and/or chloroquine, and its derivatives, alone or in combinations thereof in human plasma required for treating and/or preventing rheumatoid arthritis and/or malaria and/or lupus erythematosus and/or SARS CoV infection and/or prophyra cutanea tarda. Therapeutically effective active agent Hydroxychloroquine and/or chloroquine, and/or its derivatives dosages refers to the therapeutic concentration of in human plasma required for treating and/or preventing rheumatoid arthritis and/or malaria and/or lupus erythematosus and/or SARS CoV infection and/or prophyra cutanea tarda. Furthermore, the precise therapeutic effective dose of Hydroxychloroquine and/or chloroquine, and its derivatives in the transdermal formulation or topical formulation or transdermal delivery system can be determined by those skilled in the art based on factors such as but not limited to the patient's condition etc. The transdermal formulation or topical formulation or transdermal delivery system will be available in different dosage strengths and patch sizes in order to achieve optimum therapeutic outcome based on patient's requirement.

In yet another embodiment, the transdermal formulation and/or topical formulation and/or transdermal delivery system of the disclosure may deliver at least therapeutic effective dose of Hydroxychloroquine and/or chloroquine and derivatives of its. Therapeutically effective doses of active agent Hydroxychloroquine and/or chloroquine, and its derivatives refers to the therapeutic concentration of active agent in human plasma required for the treatment and/or prevention and/or control of rheumatoid arthritis and/or malaria and/or lupus erythematosus and/or SARS CoV infection and/or prophyra cutanea tarda.

The transdermal formulation or transdermal patch of active agent Hydroxychloroquine and/or chloroquine and its derivatives can be applied to the skin surface in any of the following dosage regimens such as once in a day, once in two days, once in three days, once in four days, once in five days, once in six days, once in a week, once in a range of from about 8 to about 13 days, once in two weeks, or once in 15 days.

### Example 12

Pressure sensitive adhesive Formulation:

| Ingredients | % W/W |
|---|---|
| Active component | 0.1%-30% |
| Solvent | 1%-40% |
| Permeation Enhancers | 1%-40% |
| Pressure sensitive adhesive | 20%-90% |
| Polymers | 2%-50% |

### The Present Formulation is not Deemed to be Limited Thereto.

While the disclosure has been described in detail and with reference to specific examples thereof, it will be apparent to one skilled in the art that various changes and modifications can be made therein without departing from the spirit and scope thereof.

### REFERENCES

1. Doan T., et.al., “Rheumatoid Arthritis: An overview of New and Emerging Therapies”, J Clin Pharmacol, 2005, 45, 751-762
2. Crowson A. N., et.al, “The cutaneous pathology of lupus erythematosus: A review”, J Cutan Pathol, 2001, 28, 1-23.
3. https://rarediseases.org/rare-diseases/porphyria-cutanea-tarda/#:˜:text-The %20disorder %20usuallv %20dcvclops %20after,most %20common %2 0form %20 of % 20porphyria
4. https://porptyriafoundation.org/for-patients/types-of-porhyria/PCT/
5. Yao X, et.al. 2020. In vitro antiviral activity and projection of optimized dosing of hydroxychloroquine for the treatment of severe acute respiratory syndrome coronavirus 2 (SARS-CoV02). Clin Infect Dis.:71(15), 732-739
6. FDA Review Reverence ID:4047416. Plaquenil Hydroxychloroquine Sulfate Tablets, U1SP
7. Carmichael et.al. 2003. Population Pharmacokinetics of Hydroxychloroquine in patient with Rheumatoid Arthritis. The Drug. Monit:25, 671-681
8. Morita et al 2016. Population Pharmacokinetics of Hydroxychloroquine in Japanese Patients with Cutaneous or Systemic lupus Erythematosus. Ther Drug Monit: 38, 259-267.
9. https://pubchem.ncbi.nlm.nih.pov/conpound/3652
10. Tett SE et.al. 1989. Bioavailability of Hydroxychloroquine tablets in healthy volunteers.

Br J Clin Pharmacol: 27, 771-779
11. Plaquenil [package insert] (2017) Concordia Pharmaceuticals Inc., St. Michael, Barbados
12. Tett SE et.al. 1993. Concentration-effect relationship of hydroxychloroquine in rheumatoid arthritis-a cross sectional study. J Rhematol.:20, 1874-1879
13. McLachlan AJ et.al. 1993. Plasma Protein binding of the enantiomers of hydroxychloroquine and metabolites. Eur J Clin Pharmacol:44, 481-484
14. Maisonnasse P et.al. 2020. Hydroxychloroquine in the treatment and prophylaxis of SARS-CoV-2 Infection in non-human primates. (Pre-Print) (https://www.researchsquare.com/article/rs-27223/v I)
15. Sinha N, et.al. 2020. Hydroxychloroquine and COVID-19. Postgrad Med J.:96(1139), 550-555
16. Jia HP et.al. 2005. ACE2 receptor expression and severe acute respiratory syndrome coronavirus infection depend on differentiation of human airway epithelia. J.

Virol.:79(23), 14614-14621
17. Fantini J et.al. 2020. Structural and molecular modelling studies revel a new mechanism of action of chloroquine and hydroxychloroquine against SARS-CoV-2 infection. Tnt J Antimicrob Agents:55(5). 105960
18. Gbinigie K et.al. 2020. Should chloroquine and hydroxychloroquine be used to treat COVID-19? A rapid review. BJGP Open:4(2):bjgpopen20X101069
19. Schrezenmeicr E. et.al. 2020. Mechanism of action of hydroxychloroquine and chloroquine: implications for rheumatology. Nat. Rev. Rheumatol.:16(3), 155-166
20. Chatre C. et.al. 2018. Cardiac complications attributed to chloroquine and hydroxychloroquine: a systemic review of the literaturc. Drug Saf.:41(10), 919-931
21. Frustaci A, et. al. 2012. Inhibition of cardiomyocyte lysosomal activity in hydroxychloroquine cardiomyopathy. Int J. Cardiol:157(1), 117-119
22. Yogasundaram H. et.al. 2014. Hydroxychloroquine-induced cardiomyopathy: Case report, pathophysiology, diagnosis, and treatment. Can J Cardiol: 30(12), 1706-1715

While the invention has been described in detail and with reference to specific examples thereof, it will be apparent to one skilled in the art that various changes and modifications can be made therein without departing from the spirit and scope thereof.

1. A Transdermal drug delivery system (TDDS) for administration of Hydroxychloroquine and/or Chloroquine comprising:
an active substance area or reservoir comprises a pharmaceutical composition comprising Hydroxychloroquine and/or Chloroquine and at least one excipient;
an impermeable backing layer;
optionally, a releasing membrane, which is covered by a detachable backing layer.

2. The TDDS according to claim 1, wherein the active substance area or reservoir is configured as a polymer matrix system, a liquid system, a gel system, or a pressure sensitive adhesive system.

3. The TDDS according to claim 1, wherein the active substance reservoir is constructed as a pouch-shaped system

4. The TDDS according to claim 1, wherein the active substance reservoir is a preparation selected from the group consisting of flowable, viscous, semi-solid, gel-like, liquid preparation, solution, dispersion, suspension, and emulsion.

5. The TDDS according to claim 1 wherein the active substance reservoir is confined on the skin facing side by an active substance permeable membrane and on the opposite side from the skin by an active substance impermeable layer.

6. The TDDS according to claim 1, comprising an active substance permeable membrane which modifies or controls the rate of active substance release.

7. The TDDS according to claim 1, characterized in that the Hydroxychloroquine and/or chloroquine containing area is a single-, double-, or multilayered active substance matrix.

8. The TDDS according to claim 1 further comprising an adhesive so that it may be applied as a plaster or bandage.

9. The TDDS according to claim 1 wherein the active substance is a matrix selected from the group consisting of natural polymers, polysaccharides, agar, alginic acid and derivatives, cassia tora, collagen, gelatin, gellum gum, guar gum, pectin, potassium cargeenan, sodium carageenan, tragacanth, xantham, gum copal, chitosan, resin, semisynthetic polymers, cellulose, methylcellulose, ethyl cellulose, carboxymethyl cellulose, hydroxylpropyl cellulose, hydroxypropylmethyl cellulose, synthetic polymers, carboxyvinyl polymers, carbomers, carbopol 940, carbopol 934, carbopol 971p NF, polyethylene, clays, silicates, bentonite, silicon dioxide, polyvinyl alcohol, acrylic polymers (eudragit), acrylic acid esters, polyacrylate copolymers, polyacrylamide, polyvinyl pyrrolidone homopolymer, polyvinyl pyrrolidone copolymers, PVP, Kollidon 30, poloxamer, isobutylene, ethyl vinyl acetate copolymers, natural rubber, synthetic rubber, pressure sensitive adhesives, silicone polymers, bio psa 4302, bio-psa 4202, acrylic pressure sensitive adhesives, duro-tak 87-2156, duro-tak 387-2287, duro-tak 87-9301, duro-tak 387-2051, polyisobutylene, polyisobutylene low molecular weight, polyisobutylene medium molecular weight, polyisobutylene 35000 mw, acrylic copolymers, rubber based adhesives, hot melt adhesives, styrene-butadiene copolymers, bentonite, all water and/or organic solvent swellable polymers and combinations thereof.

10. The TDDS according to claim 1, wherein the active substance reservoir contains a fiber material, a woven fabric, or a nonwoven fabric, to which the active substance is adsorbed.

11. The TDDS according to claim 1, can deliver 1-40 mg/day Hydroxychloroquine and/or chloroquine through the skin to the blood in a subject, and which can produce up to 2000 ng/ml plasma concentration.

12. The TDDS according to claim 1, wherein the Hydroxychloroquine and/or chloroquine is present in a concentration in the range of from 0.1-50 wt % relative total mass of the active substance reservoir.

13. The TDDS according to claim 1, wherein the Hydroxychloroquine and/or chloroquine is present in a concentration in the range of from 1-30 wt % relative total mass of the active substance reservoir.

14. The TDDS according to claim 1, wherein the Hydroxychloroquine and/or chloroquine is present in a concentration in the range of from 1-20 wt %, relative total mass of the active substance reservoir.

15. The TDDS according to claim 1, wherein Hydroxychloroquine and/or chloroquine is present in the active substance reservoir either in dissolved or suspended state.

16. The TDDS according to claim 1, wherein the active substance reservoir contains at least one solubilizer in an amount of from 1 to 99 wt % relative to the total weight of the active substance reservoir.

17. The TDDS according to claim 1, wherein the active substance reservoir contains at least one solubilizer in an amount of from 5 to 70 wt % relative to the total weight of the active substance reservoir.

18. The TDDS according to claim 16, wherein the solubilizer is selected from the group consisting of methanol, ethanol, isopropyl alcohol, butanol, propanol, polyhydric alcohols, glycols, propylene glycol, polyethylene glycol, dipropylene glycol, hexylene glycol, butyene glycol, glycerine, derivative of glycols, pyrrolidone, N methyl 2-pyrrolidone, 2 pyrrolidone, sulfoxides, dimethyl sulfoxide, decylmethylsulfoxide, dimethylisosorbide, mineral oils, vegetable oils, sesame oil water, polar solvents, semi polar solvents, non polar solvents, volatile chemicals, ethanol, propanol, ethyl acetate, acetone, methanol, dichloromethane, chloroform, toluene, IPA, hexane, acids, acetic acid, lactic acid, levulinic acid, bases, pentane, dimethylformamide, butane, lipids, and combinations thereof.

19. The TDDS according to claim 1, wherein the active substance reservoir contains at least one permeation-enhancing agent, in an amount of from 0.1 to 50 wt % relative to the total weight of the active substance reservoir.

20. The TDDS according to claim 1, wherein the active substance reservoir contains at least one permeation-enhancing agent, in an amount of from 1 to 25 wt % relative to the total weight of the active substance reservoir.

21. The TDDS according to claim 19 where in the permeation-enhancing agent is selected and is selected from the group consisting of dimethylsulfoxide, dimethylacetamide, dimethylformamide, decylmethylsulfoxide, dimethylisosorbide, azone, pyrrolidones, N-methyl-2-pyrrolidone, 2-pyrrolidon, esters, fatty acid esters, propylene glycol monolaurate, butyl ethanoate, ethyl ethanoate, isopropyl myristate, isopropyl palmitate, methyl ethanoate, lauryl lactate, ethyl oleate decyl oleate, glycerol monooleate, glycerol monolaurate, lauryl laurate, fatty acids, capric acid, caprylic acid, lauric acid, oleic acid, myristic acid, linoleic acid, stearic acid, palmitic acid, alcohols, fatty alcohols, glycols, oleyl alcohol, nathanol, dodecanol, propylene glycol, glycerol, ethers, alcohol, diethylene glycol monoethyl ether, urea, triglycerides, triacetin, polyoxyethylene fatty alcohol ethers, polyoxyethylene fatty acid esters, esters of fatty alcohols, essential oils, surfactant type enhancers, brij, sodium lauryl sulfate, tween, polysorbate, terpene, terpenoids, and combinations thereof.

22. The pharmaceutical composition of claim 1 formulated as transdermal liquid formulation, transdermal semisolid formulation, or transdermal polymer matrix formulation, transdermal adhesive matrix formulation, film forming gel formulation, or film forming spray formulation

23. The TDDS according to claim 1, characterized in that during application period of the TDDS an irritation score is at a minimum 0 and maximum 3.

24. A method of treating rheumatoid arthritis, malaria, lupus erythematosus, SARS CoV-2 Infection, and porphyria Cutanea tarda by administration of a Hydroxychloroquine and/or chloroquine-containing TDDS according to claim 1 to a patient, thereby treating rheumatoid arthritis, malaria, lupus erythematosus, SARS CoV-2 Infection, and porphyria Cutanea tarda.

25. A method of treating and/or preventing rheumatoid arthritis comprising:
selecting a patient in need of such treatment and/or prevention;
applying to the skin of the patient a TDDS as set forth in claim 1;
thereby treating and/or preventing the rheumatoid arthritis.

26. A method of treating and/or preventing lupus erythematosus comprising:
selecting a patient in need of such treatment and/or prevention;
applying to the skin of the patient a TDDS as set forth in claim 1;
thereby treating and/or preventing the anxiety.

27. A method of treating and/or preventing malaria comprising:
selecting a patient in need of such treatment and/or prevention;
applying to the skin of the patient a TDDS as set forth in claim 1;
thereby treating and/or preventing the anxiety.

28. A method of treating and/or preventing SARS CoV-2 comprising:
selecting a patient in need of such treatment and/or prevention;
applying to the skin of the patient a TDDS as set forth in claim 1;
thereby treating and/or preventing the anxiety.

29. A method of treating and/or preventing Prophyria Cutanea Tarda comprising:
selecting a patient in need of such treatment and/or prevention;
applying to the skin of the patient a TDDS as set forth in claim 1;
thereby treating and/or preventing the anxiety.

30. The method according to claim 1, characterized in that the application period of the TDDS is at least 24 Hr and maximally 7 days.

31. A method of making a transdermal drug delivery system (TDDS) for administration of Hydroxychloroquine and/or chloroquine comprising:
providing an active substance area or reservoir;
providing an impermeable backing layer;
optionally providing a releasing membrane, which is covered by a detachable backing layer,
wherein the active substance area or reservoir comprises a pharmaceutical composition comprising Hydroxychloroquine and/or chloroquine and at least one excipient.
